(19) United States
(12) Patent Application Publication
(10) Pub. No.: US 2008/0058765 A1
(43) Pub. Date: Mar. 6, 2008

(54) Catheter for linear and circular mapping

(76) Inventors: Pierri Jais, Pessac-Bordeaux (FR); James K. Lee, West Covina, CA (US); Robert A. Mest, Long Beach, CA (US)

Correspondence Address: CHRISTIE, PARKER & HALE, LLP, PO BOX 7068, PASADENA, CA, 91109-7068, US

(21) Appl. No.: 11/515,320

(22) Filed: Aug. 31, 2006

(51) Int. Cl. A61M 25/00

(52) U.S. Cl. 604/523

(57) ABSTRACT

A catheter is provided which are useful for mapping circular regions of or near the heart as well as linear regions extending from the tubular regions. The catheter comprises a catheter body, a flexible, generally linear mapping section and a generally circular mapping section. The generally circular mapping section is used to map electrical activity within a tubular region of or near the heart. The generally linear mapping section is used to concurrently map electrical activity of linear regions extending from the tubular region in which the generally circular mapping section is located.

### FIELD OF THE INVENTION

The present invention relates to improved mapping catheters useful for mapping electrical activity in tubular and linear regions of or near the heart.

### BACKGROUND OF THE INVENTION

Atrial fibrillation is the most common type of cardiac arrythmia and can result in various adverse effects. For example, atrial fibrillation can result in a fast and irregular cardiac rhythm which will lead to heart palpitations. In addition, atrial fibrillation can cause a deterioration in cardiac function. On average, atrial fibrillation causes a 30% decrease in cardiac output. Also, atrial fibrillation causes increased incidences of intra-cardiac thrombosis, which can lead to embolic events such as strokes. Paroxysmal or chronic atrial fibrillation is responsible for 20 to 35% of cerebro-vascular accidents (“CVA”s).

Atrial fibrillation can be treated by pulmonary vein isolation. However, such treatment proves insufficient in 30 to 50% of paroxysmal atrial fibrillation patients and in 90% of permanent atrial fibrillation patients. In many instances, linear lesions in the right and/or left atrium may be necessary in addition to pulmonary vein isolation.

Each linear lesion must be transmural and continuous with adjacent lesions to obtain a final line, which blocks electrical activity between two natural areas of block. For example, a linear lesion may be created in the Mitral isthmus in the left atrium, where the lesion extends from the Mitral annulus to the left inferior pulmonary vein. Alternatively, a linear lesion may be created on the roof of the left atrium, where the lesion connects the ostium of the superior right pulmonary vein to the left superior vein. Although making linear lesions with a radio frequency catheter is well known, these linear lesions are extremely difficult to create and assess with current catheters because they require repeated point-to-point assessments of the linear lesion.

### SUMMARY OF THE INVENTION

The invention is directed to a mapping catheter capable of simultaneously mapping a tubular region of or near the heart and a linear region extending from the tubular region. Such dual mapping capabilities enable efficient linear lesion assessment. In one embodiment, the catheter comprises an elongated catheter body, a mapping assembly and a control handle. The mapping assembly includes a generally linear mapping section and a generally circular mapping section.

The generally circular mapping section is used to map electrical activity within a tubular region of or near the heart. The generally linear mapping section is used to map electrical activity of a generally linear region extending from the tubular region. As such, both the generally circular mapping section and the generally linear mapping section carry mapping electrodes. In one embodiment, each of the generally circular mapping section and the generally linear mapping section carries ten electrodes or electrode pairs.

The generally linear mapping section is very flexible, i.e. more flexible than both the catheter body and generally circular mapping section. The flexibility of the generally linear mapping section enables the section to flop over and contact a linear region of tissue upon exertion of distal force on the catheter shaft. As such, the flexibility of the generally linear mapping section prevents the generally circular mapping section from becoming dislodged from its position within the tubular region. In that regard, the generally circular mapping section acts as a mechanical anchor for the generally linear mapping section.

### BRIEF DESCRIPTION OF THE DRAWINGS

These and other features and advantages of the present invention will be better understood by reference to the following detailed description when considered in conjunction with the accompanying drawings wherein:

FIG. 1 is an elevated side view of a catheter according to one embodiment of the invention;

FIG. 2a is an exaggerated side cross-sectional view of a catheter body according to one embodiment of the present invention taken along a first diameter, including the junction between the catheter body and generally linear mapping section of the mapping assembly;

FIG. 2b is an exagerrated side cross-sectional view of the catheter body of FIG. 2a taken along a second diameter different from the first diameter of FIG. 2a;

FIG. 2c is an exagerrated longitudinal cross-sectional view of the catheter body of FIG. 2a taken along line 2c-2c;

FIG. 3a is an exaggerated side cross-sectional view of the generally linear mapping section of a mapping assembly according to one embodiment of the present invention taken along a first diameter, including the junction between the generally linear mapping section and the generally circular mapping section;

FIG. 3b is an exaggerated side cross-sectional view of the linear mapping assembly of the catheter of FIG. 3a taken along a second diameter different from the first diameter of FIG. 3a;

FIG. 3c is an exagerrated longitudinal cross-sectional view of the linear mapping section of the catheter of FIG. 3a taken along line 3c-3c;

FIG. 3d is an exaggerated longitudinal cross-sectional view of the generally circular mapping section of the catheter of FIG. 3a taken along line 3d-3d;

FIG. 4a is an exagerrated side cross-sectional view of the generally linear mapping section of a mapping assembly according to another embodiment of the present invention taken along a first diameter, including the junction of the generally linear mapping section and the generally circular mapping section;

FIG. 4b is an exagerrated side cross-sectional view of the linear mapping assembly of the catheter of FIG. 4a taken along a second diameter different from the first diameter of FIG. 4a;

FIG. 4c is an exaggerated longitudinal cross-sectional view of the junction of the generally linear mapping section and the generally circular mapping section of FIGS. 4a and 4b taken along line 4c-4c;

FIG. 5a is a side view of a circular mapping section according to one embodiment of the present invention in a clockwise formation;

FIG. 5b is a side view of a circular mapping section according to one embodiment of the present invention in a counterclockwise formation rotated 90° relative to the circular mapping section depicted in FIG. 5a;

FIG. 5c is a schematic view of a circular mapping section according to one embodiment of the present invention;

FIG. 5d is a schematic view of a circular mapping section according to one embodiment of the invention depicting the relationship between a first and a last electrode;

FIG. 6 is an exaggerated side view of a linear mapping section according to one embodiment of the present invention; and

FIG. 7 is an exaggerated schematic depicting a catheter in a pulmonary vein and left atrium according to one embodiment of the present invention.

### DETAILED DESCRIPTION OF THE INVENTION

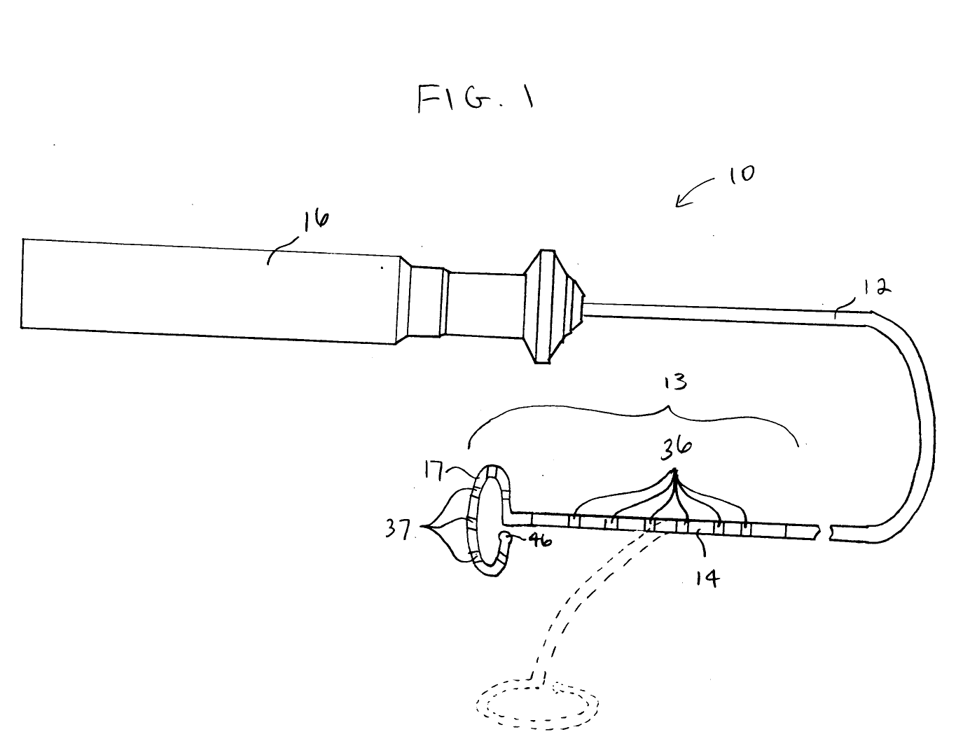

In one embodiment of the present invention, a catheter has a mapping assembly at its distal end. The mapping assembly includes a generally linear mapping section and a generally circular mapping section. As shown in FIG. 1, the catheter 10 generally comprises an elongated catheter body 12 having proximal and distal ends, a mapping assembly 13 at the distal end of the catheter body 12 including a generally linear mapping section 14 and a generally circular mapping section 17, and a control handle 16 at the proximal end of the catheter body 12.

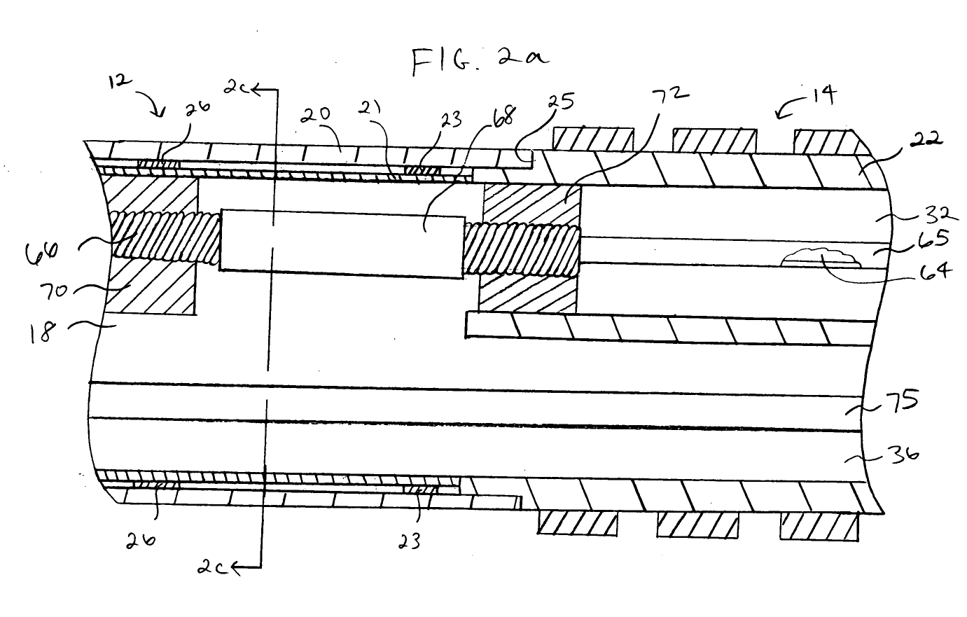

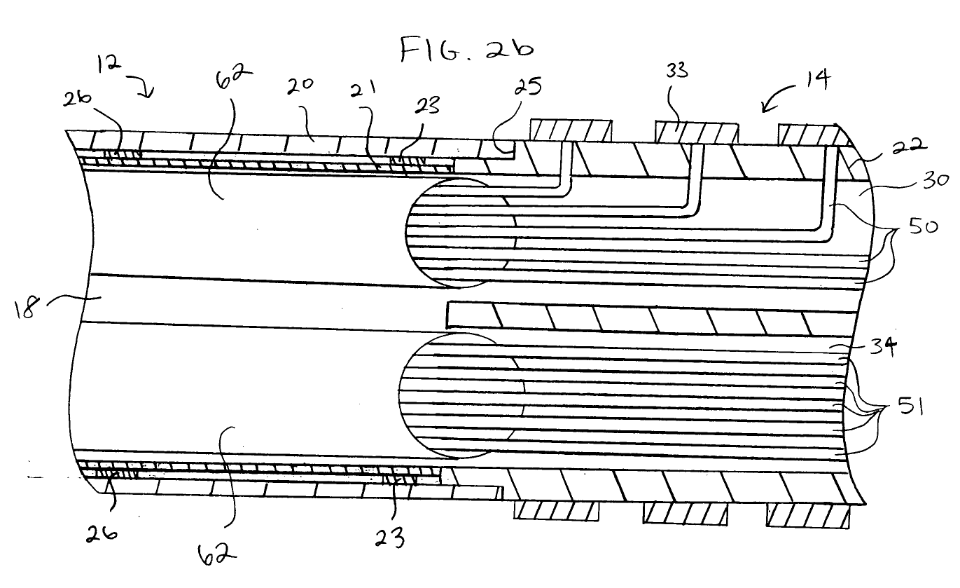

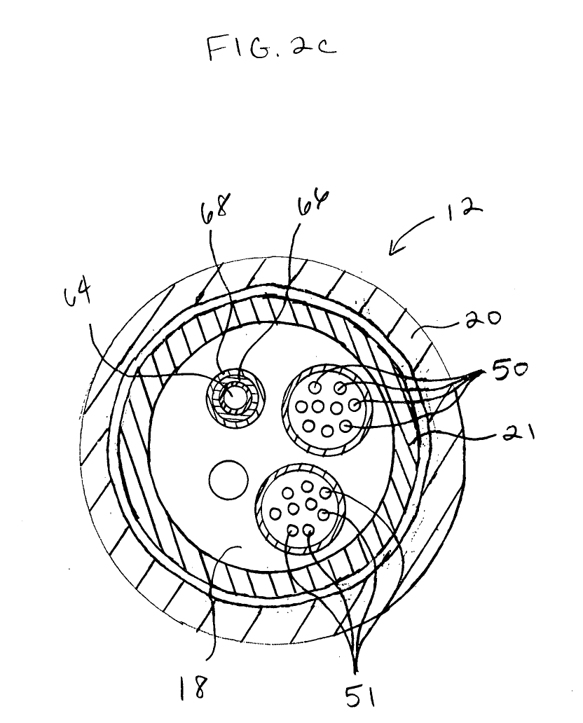

As shown in FIGS. 2a, 2b and 2c, the catheter body 12 comprises an elongated tubular construction having a single, axial or central lumen 18. The catheter body 12 is flexible, i.e., bendable, but substantially non-compressible along its length. The catheter body 12 can be of any suitable construction and made of any suitable material. One exemplary construction comprises an outer wall 20 made of an extruded plastic, e.g. polyurethane or PEBAX. The outer wall 20 may comprise an imbedded braided mesh of stainless steel or the like to increase torsional stiffness of the catheter body 12 so that when the control handle 16 is rotated, the mapping assembly 13 will rotate in a corresponding manner.

Extending through the single lumen 18 of the catheter body 12 are components, for example lead wires, puller wires, compression coils through which puller wires extends, and an electromagnetic sensor cable. A single lumen catheter body may be preferred over a multi-lumen body because it has been found that the single lumen body permits better tip control when rotating the catheter. The single lumen permits the various components such as the lead wires and the puller wire surrounded by the compression coil to float freely within the catheter body. If such components were restricted within multiple lumens, they tend to build up energy when the handle is rotated. This built-up energy results in a tendency of the catheter body to rotate back if, for example, the handle is released. The built-up energy may also force the catheter to flip over if it is bent around a curve. Either of these consequences are undesirable performance characteristics.

The outer diameter of the catheter body 12 is not critical, but in one embodiment is no more than about 8 french. In another embodiment, the outer diameter of the catheter body 12 is about 6.5 french. Likewise, the thickness of the outer wall 20 is not critical, but is thin enough so that the central lumen 18 can accommodate the aforementioned puller wire, lead wires, and any other desired wires, cables or tubes. If desired, the inner surface of the outer wall 20 may be lined with a stiffening tube 21, which can be made of any suitable material, such as polyimide or nylon. The stiffening tube 21, along with the braided outer wall 20, provides improved torsional stability while at the same time minimizing the wall thickness of the catheter, thus maximizing the diameter of the central lumen 18. The outer diameter of the stiffening tube 21 is about the same as or slightly smaller than the inner diameter of the outer wall 20. Polyimide tubing may be used for the stiffening tube 21 because it may be very thin walled while still providing very good stiffness. This maximizes the diameter of the central lumen 18 without sacrificing strength and stiffness.

As noted above, the mapping assembly 13 includes a generally linear mapping section 14 and a generally circular mapping section 17. The generally circular mapping section 17 of the mapping assembly 13 may be used to map a tubular region of or near the heart, for example a pulmonary vein. However, mapping of linear regions extending from such tubular regions may also be necessary. Accordingly, the generally linear mapping section 14 may be used to map such linear regions, for example the Mitral isthmus or the roof of the left atrium. The generally linear mapping section 14 can map these linear regions concurrently with the mapping of the tubular region by the generally circular mapping section 17. In order to do so, however, the generally linear mapping section 14 has a very flexible construction such that it can be forced against the linear region by simply pushing the catheter shaft distally after placement of the generally circular mapping section 17 in the tubular region. By this design, the circular mapping section 17 acts as an anchor for the linear mapping section 14 and the flexibility of the linear mapping section 14 prevents the circular mapping section 17 from becoming dislodged upon exertion of distal force on the catheter shaft. Rather, once the circular mapping section 17 is placed in the tubular region, the exertion of distal force on the catheter shaft causes the flexible linear mapping section 14 to flop over and contact the linear region extending from the tubular region. The linear mapping section 14 can be used to map any linear radial pattern extending from the tubular region in which the circular mapping section 17 is located.

The generally linear mapping section 14 comprises a section of flexible tubing 22 having four lumens. The flexible tubing 22 is made of a suitable non-toxic material that is more flexible than both the catheter body 12 and the generally circular mapping section 17. A suitable material for the tubing 22 is braided polyurethane, i.e., polyurethane with an embedded mesh of braided stainless steel or the like. The flexible tubing 22 is floppy such that upon exertion of distal force on the catheter shaft, the tubing 22 will flop over. As such, the flexible tubing 22 generally comprises a polyurethane having a durometer ranging from about 40 to 65D. In one embodiment, the polyurethane comprises a compound mixture of 55D polyurethane, 65D polyurethane and 80A polyurethane. The resulting tubing is slightly more flexible than a 55D polyurethane. Although the tubing 22 is described as braided polyurethane, it is understood that any suitable plastic material can be used so long as the material has generally the same flexibility and is biocompatible.

As noted above, the flexible tubing 22 of the generally linear mapping section includes four lumens. In addition, the outer diameter of the generally linear mapping section 14 is not critical but is slightly smaller than that of the catheter body 12, measuring about 6.5 french. The smaller diameter of the quad lumen tubing 22 contributes added flexibility to the generally linear mapping section and enhances the section's ability to conform to the intended anatomical regions.

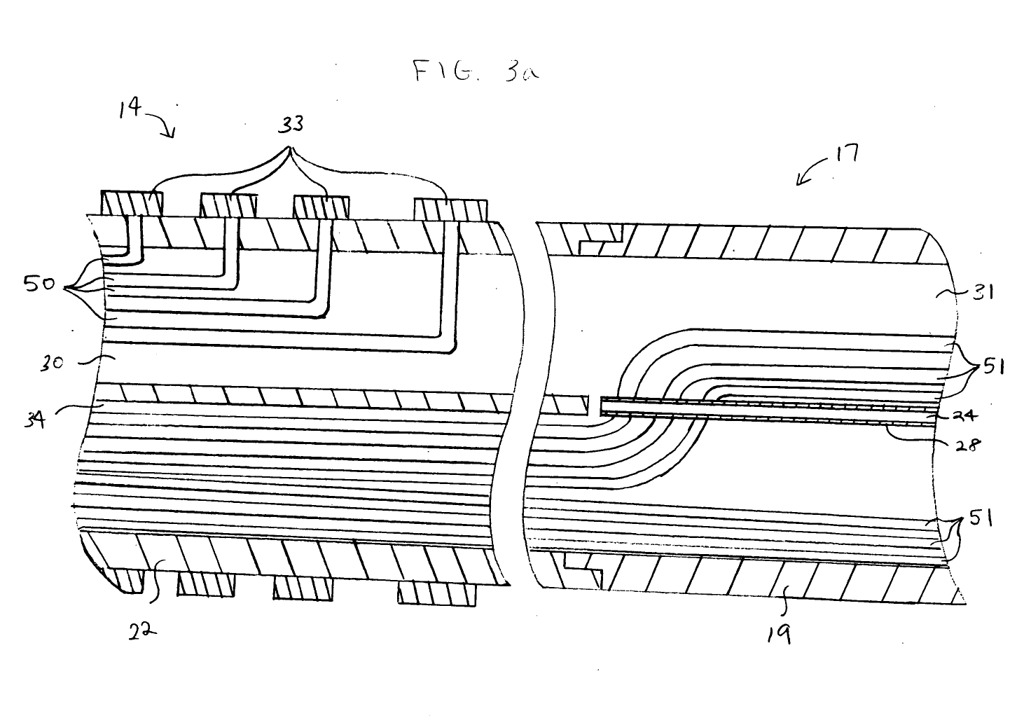

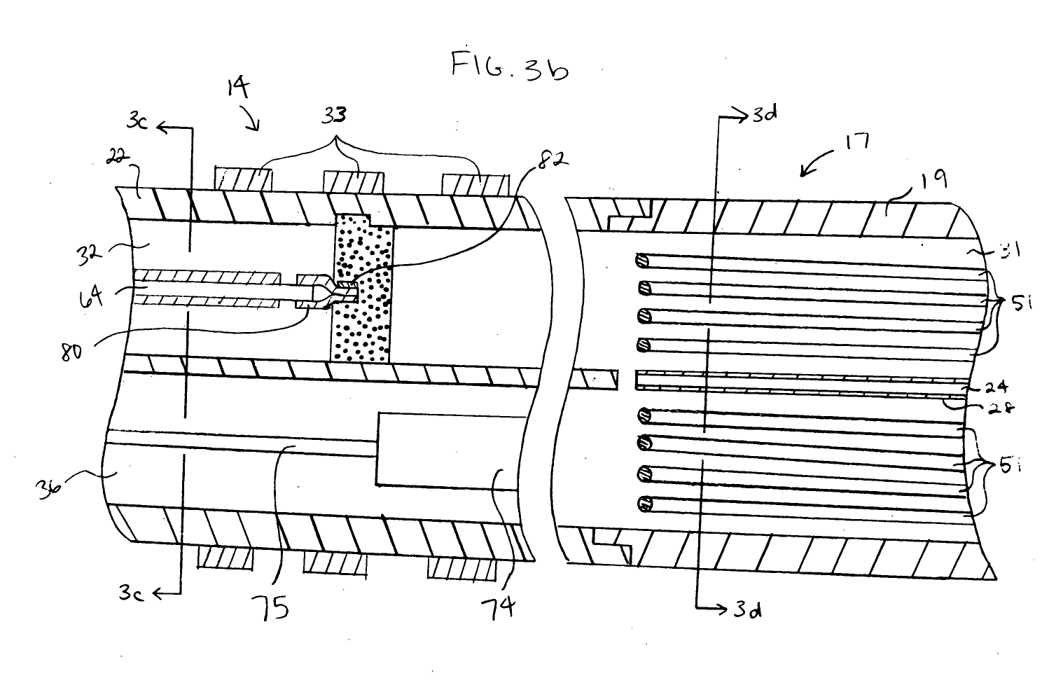

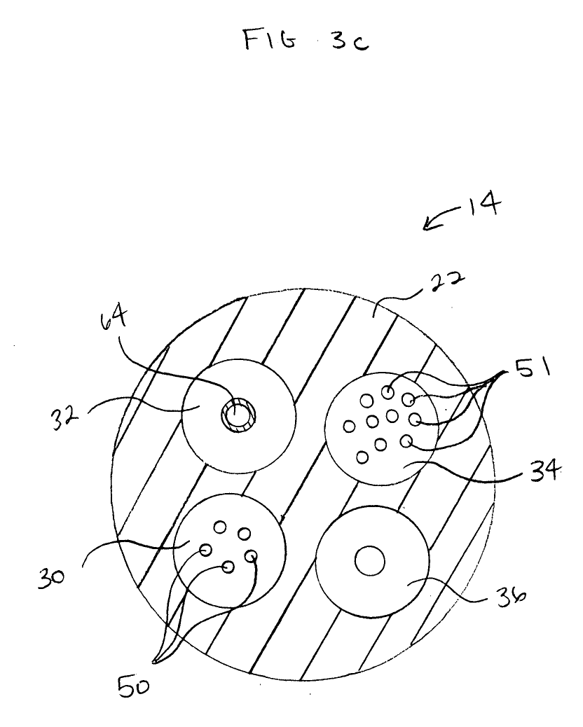

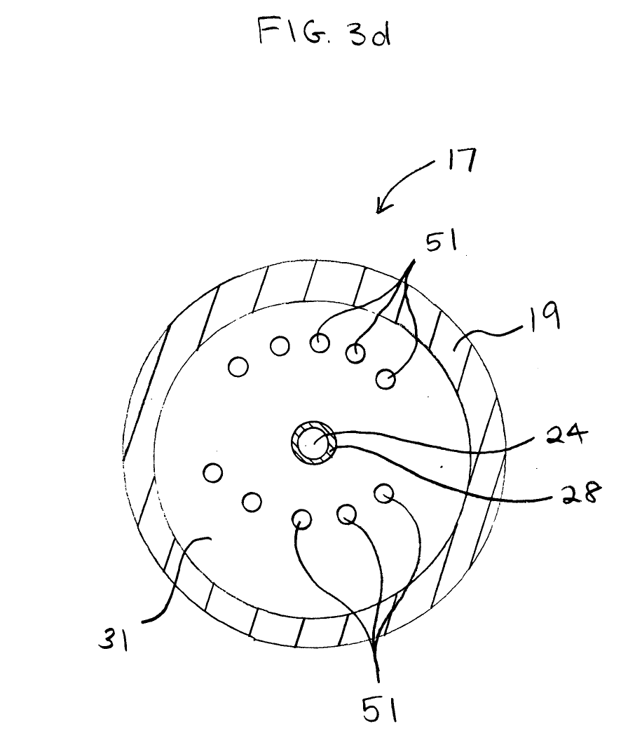

As shown in FIGS. 2b and 3c, the first lumen 30 of the tubing 22 carries electrode lead wires 50 from electrodes mounted on the generally linear mapping section 14 and the third lumen 34 carries electrode lead wires 51 from electrodes mounted on the generally circular mapping section 17. As shown in FIGS. 2a and 3c, the second lumen 32 carries a puller wire 64 and the fourth lumen 36 carries an electromagnetic sensor cable. Although illustrated as carrying an electromagnetic sensor cable, it is understood that the fourth lumen 36 may alternatively remain empty or may carry a second puller wire, or any other desired wires, cables or tubes. The size of each lumen is not critical, but is sufficient to house the lead wires, puller wire, etc.

The useful length of the catheter, i.e., that portion that can be inserted into the body excluding the circular mapping section 17, can vary as desired. In one embodiment, the useful length ranges from about 10 cm to about 120 cm. The length of the generally linear mapping section 14 makes up a portion of the useful length, and ranges from about 6 to about 7 cm. In one embodiment, the generally linear mapping section 14 has a length ranging from about 6.2 to about 6.8 cm.

One means for attaching the catheter body 12 to the mapping assembly 13 is illustrated in FIGS. 2a and 2b. The proximal end of the generally linear mapping section 14 of the mapping assembly 13 comprises an outer circumferential notch 25 that receives the inner surface of the outer wall 22 of the catheter body 12. The mapping assembly 13 and catheter body 12 are attached by glue or the like. Before the mapping assembly 13 and catheter body 12 are attached, the stiffening tube 21 is inserted into the catheter body 12. The distal end of the stiffening tube 21 is fixedly attached near the distal end of the catheter body 12 by forming a glue joint (not shown) with polyurethane glue or the like. In one embodiment, a small distance, e.g. about 3 mm, is provided between the distal end of the catheter body 12 and the distal end of the stiffening tube 21 to permit room for the catheter body 12 to receive the notch 25 of the mapping assembly 13. If no compression coil is used, a force is applied to the proximal end of the stiffening tube 21, and while the stiffening tube 21 is under compression, a first glue joint 23 is made between the stiffening tube 21 and the outer wall 20 by a fast drying glue, e.g. cyanoacrylate. Thereafter, a second glue joint 26 is formed between the proximal ends of the stiffening tube 21 and outer wall 20 using a slower drying, but strong glue, e.g. polyurethane.

If desired, a spacer (not shown) can be located within the catheter body between the distal end of the stiffening tube (if provided) and the proximal end of the mapping assembly. The spacer provides a transition in flexibility at the junction of the catheter body and mapping assembly, which allows this junction to bend smoothly without folding or kinking. A catheter having such a spacer is described in U.S. Pat. No. 5,964,757, entitled “Steerable Direct Myocardial Revascularization Catheter,” the entire content of which is incorporated herein by reference.

At the distal end of the generally linear mapping section 14 of the mapping assembly 13 is a generally circular mapping section 17, as shown in FIGS. 3a, 3b, 3d, 4a, 4b, 4c, 5a, 5b, 5c and 5d. The generally circular mapping section 17 comprises a short section of tubing 19 having a single, central lumen 31. A support member 24 is disposed in the generally circular mapping section 17. In one embodiment, as shown in FIGS. 3a, 3b and 3d, the support member extends through the generally circular mapping assembly but does not extend into the generally linear mapping section 14.

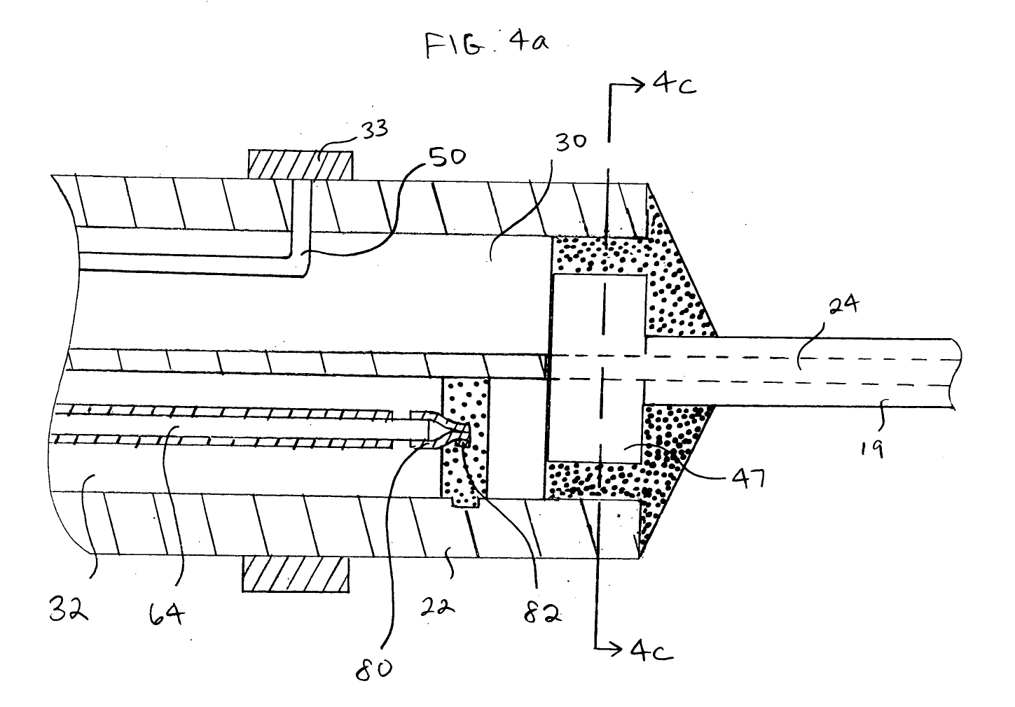

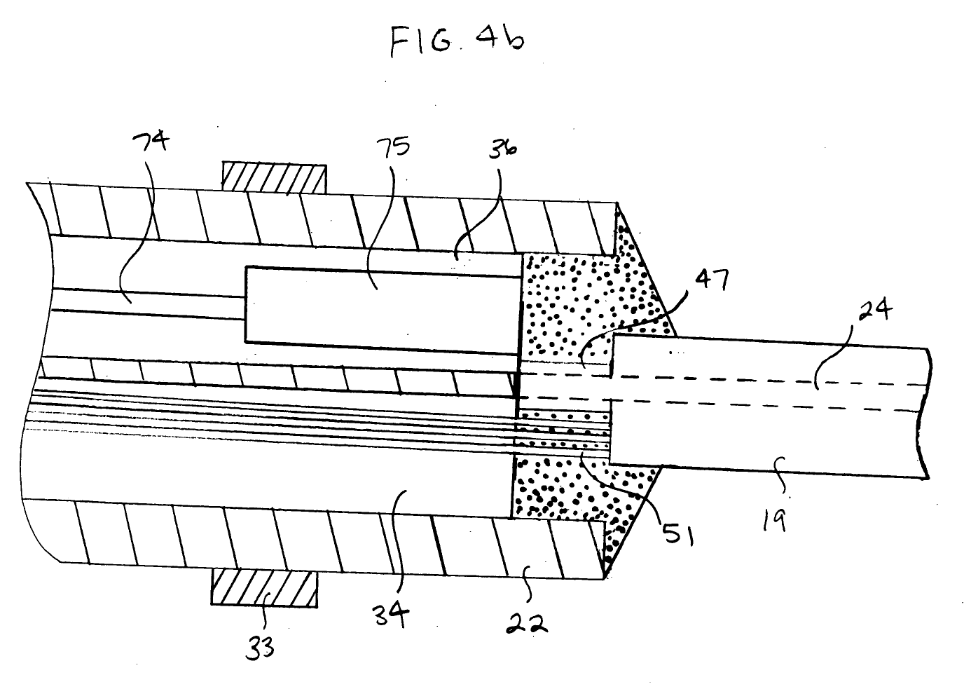

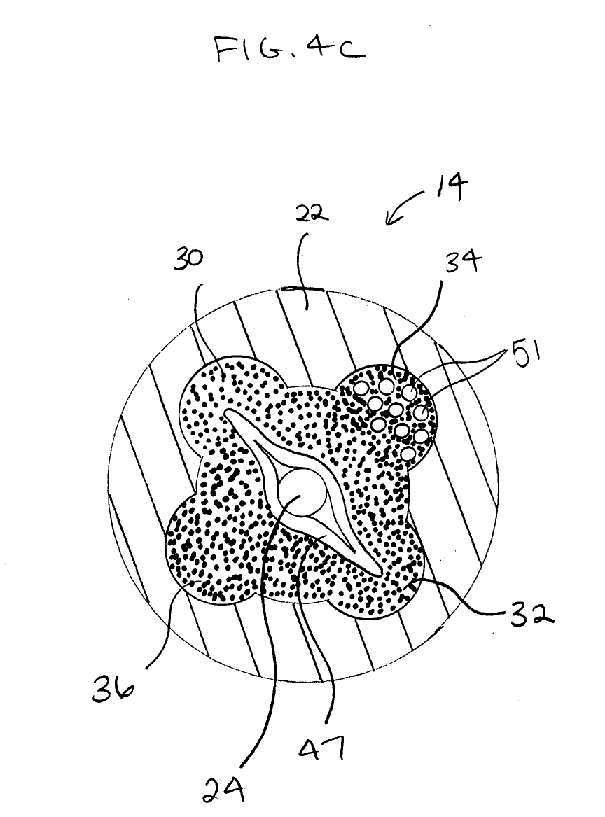

In another embodiment, as shown in FIGS. 4a, 4b and 4c, the support member 24 is anchored in the distal end of the generally linear mapping section 14. To anchor the support member 24 in the distal end of the generally linear mapping section 14, a metal hypotube 47 is crimped over a short segment of the proximal end of the support member 24. A small length of the distal end of the generally linear mapping section 14 is cored out to provide space for the crimped metal hypotube 47. The distal end of the generally linear mapping section 14 is cored out such that the lumens 30, 32, 34 and 36 of the tubing 22 open into the cored out section, as best shown in FIG. 4c. As also shown in FIG. 4c, the crimped metal hypotube 47 holding the support member 24 sits partially in the first lumen 30 and partially in the second lumen 32. Because the puller wire housed in the second lumen 32 and the lead wires housed in the first lumen 30 terminate proximal the junction of the generally linear mapping section 14 and the generally circular mapping section 17, the lumens 30 and 32 are empty within the junction, making room for the crimped metal hypotube and support member 24. Once positioned in the cored out section of the generally linear mapping section 14, the support member 24 is fixed in place by gluing the metal hypotube within the cored out section, for example with polyurethane glue. As shown, the generally circular mapping section 17 has a diameter slightly smaller than that of the generally linear mapping section 17. Accordingly, the space between the generally linear mapping section 14 and the generally circular mapping section 17 is filled with glue (e.g. polyurethane glue). This constructions prevents the generally circular mapping section from rotating relative to the generally linear mapping section and prevents the generally circular mapping section from being pulled or slipping out of the generally linear mapping section.

The generally circular shape of the circular mapping section 17 is formed from the distal end of the support member 24 covered by a non-conductive covering 28. In such an embodiment, the support member 24 is made of a material having shape-memory (i.e. a material that can be straightened or bent out of its original shape upon exertion of a force and that is capable of substantially returning to its original shape upon removal of the force). One exemplary material for the support member 24 is a nickel/titanium alloy. Such alloys typically comprise about 55% nickel and 45% titanium, but may comprise from about 54% to about 57% nickel with the balance being titanium. One such nickel/titanium alloy is Nitinol, which has excellent shape memory, together with ductility, strength, corrosion resistance, electrical resistivity and temperature stability. The non-conductive covering 28 can be made of any suitable material, and one exemplary material is a biocompatible plastic such as polyurethane or PEBAX.

The generally circular mapping assembly 17 comprises a generally straight proximal region 38, and a generally circular main region 39. The generally circular mapping assembly 17 may further comprise a generally straight distal region, as illustrated and described in U.S. Pat. No. 6,628,976, entitled “Catheter Having Mapping Assembly,” the entire content of which is incorporated herein by reference.

The proximal region 38 is mounted on the generally linear mapping section 14 of the mapping assembly 13 (as described in more detail below) so that its axis is generally parallel to the axis of the generally linear mapping section 14. In one embodiment, the proximal region 38 has an exposed length (i.e. the length not contained within the generally linear mapping section 14) ranging from about 4 mm to about 8 mm, but can vary as desired.

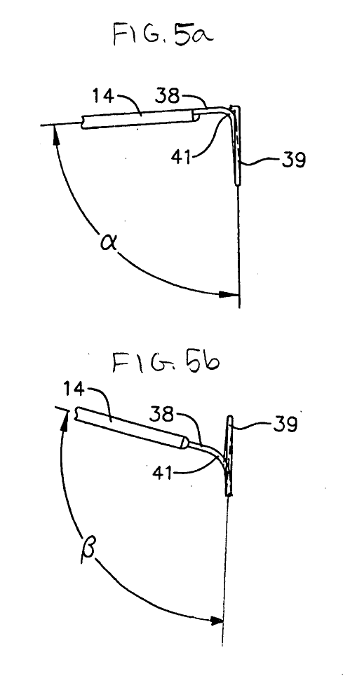

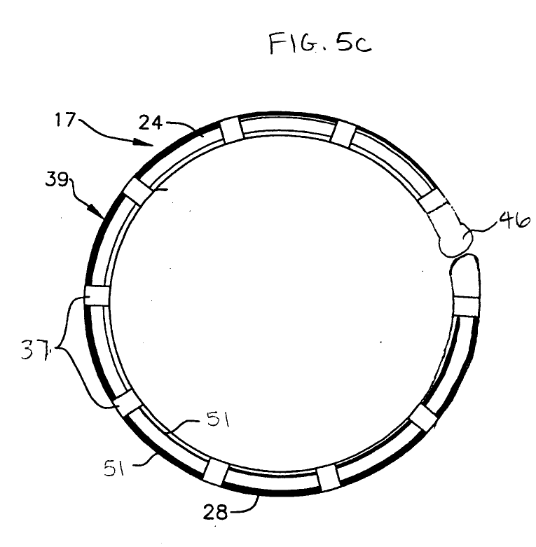

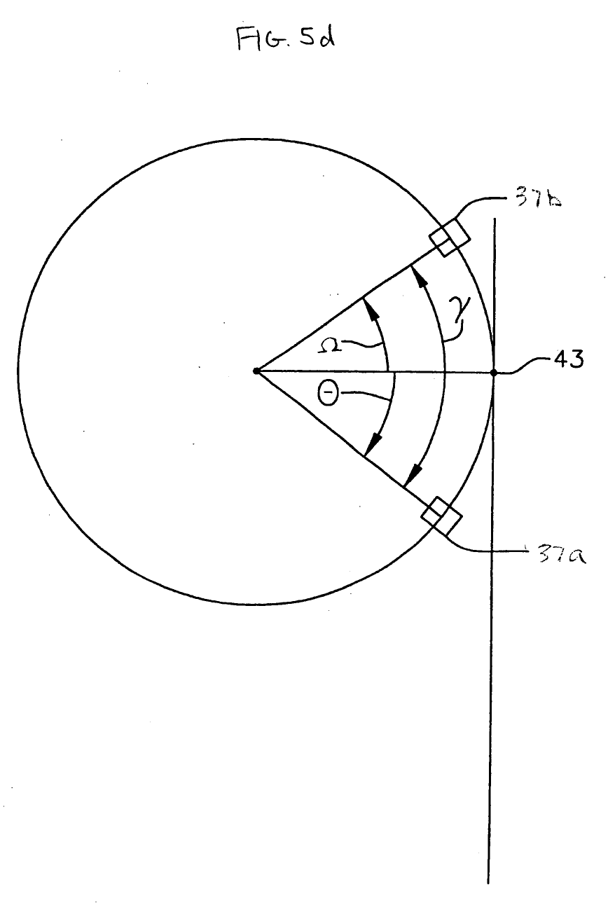

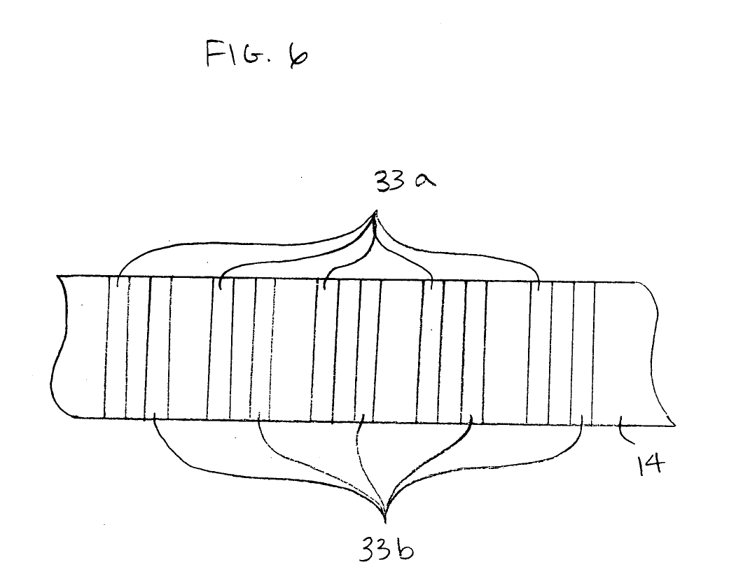

In one embodiment, the generally circular main region 39 of the generally circular mapping section 17 has an outer diameter ranging from about 10 mm to about 25 mm. In another embodiment, the generally circular main region 39 has an outer diameter ranging from about 12 mm to about 20 mm. In still another embodiment, the generally circular main region 39 has an outer diameter of about 15 mm. The transition region 41 from the straight proximal region 38 to the generally circular main region 39 may be slightly curved and formed such that, when viewed from the side with the proximal region at the top of the circular main region as shown in FIG. 5a, the proximal region (along with the generally linear mapping section 14) forms an angle α with the curved region ranging from about 75° to about 95°. In one embodiment, the angle α ranges from about 83° to about 93°. In another embodiment, the angle α is about 87°.

The generally circular main region 39 can curve in a clockwise direction, as shown in FIG. 5a, or a counterclockwise direction, as shown in FIG. 5b. When the generally circular mapping section 17 is turned 90°, as shown in FIG. 5b, so that the transition region 41 is near the center of the generally circular main region 39, the proximal region 38 (along with the generally linear mapping section 14) forms an angle β with the generally circular main region 39 ranging from about 90° to about 135°. In one embodiment, the angle β ranges from about 100° to about 110°. In another embodiment, the angle β is about 105°.

A first series of ring electrodes 33 are mounted on the generally linear mapping section (see FIGS. 1, 2a, 2b, 3a, 3b and 5) 14, and a second series of ring electrodes 37 are mounted on the generally circular main region 39 of the generally circular mapping section 17 (see FIG. 5c). The ring electrodes 33 and 37 can be made of any suitable solid conductive material, such as platinum or gold. One exemplary material comprises a combination of platinum and iridium. The ring electrodes 33 and 37 are mounted onto the tubing 22 of the generally linear mapping section 14 or the non-conductive covering 28 of the generally circular main region 39 with glue or the like. Alternatively, the ring electrodes can be formed by coating the tubing 22 of the linear mapping section 14 or the non-conductive covering 28 of the generally circular main region 39 with an electrically conductive material, such as platinum, gold and/or iridium. Such a coating can be applied by sputtering, ion beam deposition or an equivalent technique.

In one embodiment, each ring electrode 33 is mounted by first forming a hole in the tubing 22 of the generally linear mapping section. An electrode lead wire 50 is fed through the hole, and the ring electrode 33 is welded in place over the lead wire 50 and tubing 22. The proximal end of each lead wire 50 is electrically connected to a suitable connector (not shown), which is connected to a source of RF energy (not shown).

The number of ring electrodes 33 on the generally linear mapping section 14 can vary as desired. In one embodiment, however, the number of ring electrodes 33 ranges from about six to about twenty. In another embodiment, the number of ring electrodes ranges from about eight to about twelve. In yet another embodiment, the number of ring electrodes is ten. The ring electrodes 33 are approximately evenly spaced along the length of the generally linear mapping section 14, as shown in FIG. 5c. In one embodiment, a distance of approximately 6 mm is provided between the centers of the ring electrodes 33.

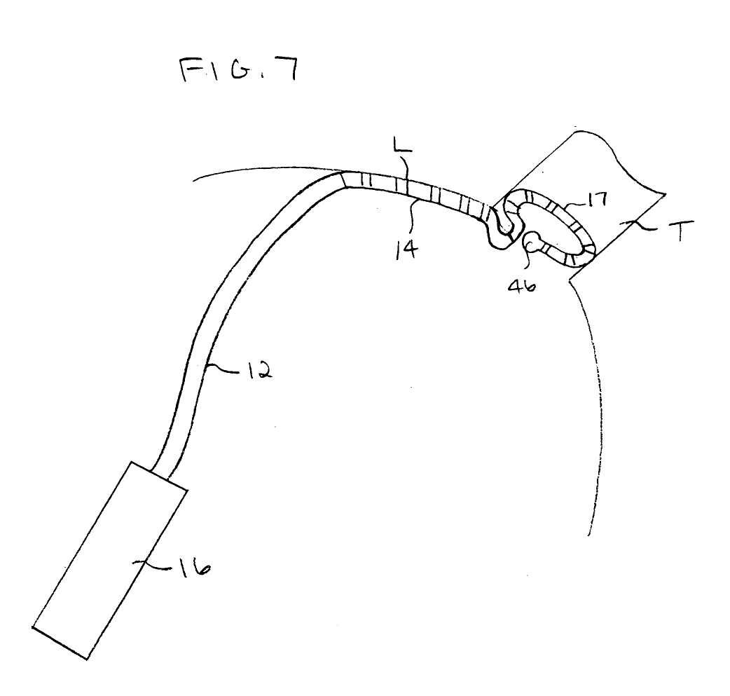

In one exemplary embodiment, the generally linear mapping section 14 comprises a plurality of electrode pairs, as shown in FIG. 6. For example, the generally linear mapping section 14 may have from three to ten electrode pairs. In another embodiment, the generally linear mapping section 14 comprises from four to six electrode pairs. In yet another embodiment, the generally linear mapping section 14 comprises five electrode pairs.

Each electrode pair comprises a proximal-most electrode 33a and a distal-most electrode 33b and the distance between the center of the proximal-most electrode 33a and the center of the distal-most electrode 33b of each electrode pair is about 1 mm. The electrode pairs are approximately evenly spaced along the length of the generally linear mapping section 14. In particular, the distance between the centers of the distal-most electrodes 33b of adjacent electrode pairs ranges from about 5 to about 7 mm. In one embodiment, the distance between the centers of the distal-most electrodes 33b of adjacent electrode pairs is about 6 mm.

In one embodiment, each ring electrode 37 is mounted on the generally circular mapping section by first forming a hole in the non-conductive covering 28 of the generally circular mapping section 17. An electrode lead wire 51 is fed through the hole, and the ring electrode 37 is welded in place over the lead wire 51 and non-conductive covering 28. The lead wires 51 extend between the non-conductive covering 28 and the support member 24. The proximal end of each lead wire 51 is electrically connected to a suitable connector (not shown), which is connected to a source of RF energy (not shown).

Like the ring electrodes 33 on the generally linear mapping section 14, the number of ring electrodes 37 on the generally circular main region 39 can vary as desired. In one embodiment, however, the number of ring electrodes ranges from about six to about twenty. In another embodiment, the number of ring electrodes ranges from about eight to about twelve. In yet another embodiment, the number of ring electrodes is ten. The ring electrodes 37 are approximately evenly spaced around the generally circular main region 39, as shown in FIG. 5c. In one embodiment, a distance of approximately 5 mm is provided between the centers of the ring electrodes 37.

As shown in FIG. 5d, one exemplary electrode arrangement includes a first electrode 37a, which is the electrode on the generally circular main region 39 closest to the generally straight proximal region 38. A second electrode 37b is provided, which is the electrode closest to the tangent point 43 defined by the distal end of the generally circular main region 39. In one embodiment, the first electrode 37a is positioned along the circumference of the generally circular main region 39 at a distance θ of no more than about 55° from the tangent point 43. In another embodiment, the distance θ is no more than about 48° from the tangent point 43. In yet another embodiment, the distance θ ranges from about 15° to about 36° from the tangent point 43. Similarly, the second electrode 37b is positioned along the circumference of the generally circular main region 39 at a distance Ω of no more than about 55° from the tangent point 43. In one embodiment, the distance Ω is no more than about 48° from the tangent point 43. In another embodiment, the distance Ω ranges from about 15° to about 36° from the tangent point 43. In one exemplary embodiment, the first electrode 37a is positioned along the circumference of the generally circular main region 39 at a distance d of no more than 100° from the second electrode 37b. In another embodiment, the distance γ is no more than about 80° from the second electrode 37b. In yet another embodiment, the distance γ ranges from about 30° to about 75° from the second electrode 37b.

In another exemplary electrode arrangement, when the generally circular main region 39 is straightened, the electrodes 37 are spaced apart from each other at a distance of about 7 to about 9 mm. The second electrode 37b is slightly larger than the remaining electrodes and is spaced apart from its adjacent electrode at a distance of about 7.5 to about 9.5 mm. The distance from the second electrode 37b to the distal end of the main region 39 ranges from about 4 to about 5 mm.

As shown in FIGS. 5a and 5b, the distal end of the generally circular main region 39 is capped, for example with polyurethane glue 46, to prevent body fluids from entering the generally circular mapping assembly 17. To cap the distal end of the main region 39, a short section at the distal end of the support member 24 is not covered by the non-conductive covering 28 and the uncovered portion is burnished to promote adhesion. The burnished end of the support member 24 and the distal end of the generally circular mapping section 17 are covered with polyurethane to form a cap 46.

The junction of the generally linear mapping section 14 and generally circular mapping section 17 is shown in FIGS. 3a and 3b. The non-conductive covering 28 is attached to the tubing 22 of the generally linear mapping section 14 by glue or the like. The lead wires 50 attached to the ring electrodes 33 on the generally linear mapping section 14 extend through the first lumen 30 of the generally linear mapping section 14, through the central lumen 18 of the catheter body 12, and out through the control handle 16. The lead wires 51 attached to the ring electrodes 37 on the generally circular mapping section 17 extend through the third lumen 34 of the generally linear mapping section 14, through the central lumen 18 of the catheter body 12, and out through the control handle 16.

The portion of the lead wires 50 and 51 extending through the central lumen 18 of the catheter body 12, control handle 16 and proximal end of the generally linear mapping section 14 are enclosed within protective sheathes 62, which can be made of any suitable material, for example polyimide. The protective sheathes 62 are anchored at their distal ends to the proximal end of the generally linear mapping section 14 by gluing them in the first lumen 30 and third lumen 34 with polyurethane glue or the like.

The puller wire 64 is provided for deflection of the mapping assembly 17. The puller wire 64 extends through the catheter body 12, is anchored at its proximal end to the control handle 16, and is anchored at its distal end to the generally linear mapping section 14. The puller wire 64 is made of any suitable metal, such as stainless steel or Nitinol, and can be coated with Teflon® or the like. The coating imparts lubricity to the puller wire 64. The puller wire 64 may have a diameter ranging from about 0.006 to about 0.010 inch.

A compression coil 66 is situated within the catheter body 12 in surrounding relation to the puller wire 64. The compression coil 66 extends from the proximal end of the catheter body 12 to the proximal end of the generally linear mapping section 14. The compression coil 66 is made of any suitable metal, preferably stainless steel. The compression coil 66 is tightly wound on itself to provide flexibility, i.e., bending, but to resist compression. The inner diameter of the compression coil 66 may be slightly larger than the diameter of the puller wire 64. The Teflon® coating on the puller wire 64 allows it to slide freely within the compression coil 66. The outer surface of the compression coil 66 is covered by a flexible, non-conductive sheath 68, e.g., made of polyimide tubing.

The compression coil 66 is anchored at its proximal end to the outer wall 20 of the catheter body 12 by proximal glue joint 70 and at its distal end to the generally linear mapping section 14 by distal glue joint 72. Both glue joints 70 and 72 may comprise polyurethane glue or the like. The glue may be applied by means of a syringe or the like through a hole made between the outer surface of the catheter body 12 and the central lumen 18. Such a hole may be formed, for example, by a needle or the like that punctures the outer wall 20 of the catheter body 12 which is heated sufficiently to form a permanent hole. The glue is then introduced through the hole to the outer surface of the compression coil 66 and wicks around the outer circumference to form a glue joint about the entire circumference of the compression coil.

The puller wire 64 extends into the second lumen 32 of the generally linear mapping section 14. In one embodiment, the puller wire 64 is anchored at its distal end to the distal end of the generally linear mapping section 14, as shown in FIG. 3b. Specifically, a T-shaped anchor is formed, which comprises a short piece of tubular stainless steel 80, e.g. hypodermic stock, which is fitted over the distal end of the puller wire 64 and crimped to fixedly secure it to the puller wire. The distal end of the tubular stainless steel 80 is fixedly attached, e.g. by welding, to a cross-piece 82 formed of stainless steel ribbon or the like. The cross-piece 82 sits beyond the distal end of the second lumen 32. The cross-piece 82 is larger than the lumen opening and, therefore, cannot be pulled through the opening. The distal end of the second lumen 32 is then filled with glue or the like, e.g. polyurethane glue. Within the second lumen 32 of the generally linear mapping section 14, the puller wire 64 extends through a plastic, e.g. Teflon®, puller wire sheath 65, which prevents the puller wire 64 from cutting into the wall of the generally linear mapping section 14 when the mapping assembly 13 is deflected.

Longitudinal movement of the puller wire 64 relative to the catheter body 12, which results in deflection of the mapping assembly 13, is accomplished by suitable manipulation of the control handle 16. Examples of suitable control handles for use in the present invention are disclosed, for example, in U.S. Pat. Nos. Re 34,502 and 5,897,529, the entire contents of which are incorporated herein by reference.

The catheter 10 may further comprise an electromagnetic sensor 74 mounted in the generally linear mapping section 14, as shown in FIGS. 3b and 3c. The electromagnetic sensor 74 is connected to an electromagnetic sensor cable 75, which extends through the fourth lumen of the generally linear mapping section 14. From the generally linear mapping section 14, the electromagnetic sensor cable 75 extends through the central lumen 18 of the catheter body and out through the control handle 16. The electromagnetic sensor cable 75 then extends out the proximal end of the control handle 16 within an umbilical cord (not shown) to a sensor control module (not shown) that houses a circuit board (not shown). The electromagnetic sensor cable 75 comprises multiple wires encased within a plastic covered sheath. In the sensor control module, the wires of the electromagnetic sensor cable 75 are connected to the circuit board. The circuit board amplifies the signal received from the electromagnetic sensor 74 and transmits it to a computer in a form understandable by the computer. Because the catheter is designed for a single use only, the circuit board may contain an EPROM chip which shuts down the circuit board approximately 24 hours after the catheter has been used. This prevents the catheter, or at least the electromagnetic sensor from being used twice.

Suitable electromagnetic sensors for use with the present invention are described, for example, in U.S. Pat. Nos. 5,558,091, 5,443,489, 5,480,422, 5,546,951, 5,568,809 and 5,391,199, the entire contents of which are incorporated herein by reference. One exemplary electromagnetic sensor 74 has a length of from about 6 mm to 7 mm and a diameter of about 1.3 mm.

In use, a suitable guiding sheath is inserted into the patient with its distal end positioned at a desired mapping location. An example of a suitable guiding sheath for use in connection with the present invention is the Preface™ Braiding Guiding Sheath, commercially available from Biosense Webster, Inc. (Diamond Bar, Calif.). The distal end of the sheath is guided into one of the atria. A catheter in accordance with the present invention is fed through the guiding sheath until its distal end extends out of the distal end of the guiding sheath. As the catheter is fed through the guiding sheath, the mapping assembly 13 is straightened to fit through the sheath. Once the distal end of the catheter is positioned at the desired mapping location, the guiding sheath is pulled proximally, allowing the deflectable mapping assembly 13 to extend outside the sheath, and the mapping assembly 13 returns to its original shape due to the shape-memory of the support member 24 in the generally circular mapping section 17. The generally circular mapping section 17 of the mapping assembly 13 is then inserted into a pulmonary vein or other tubular region (such as the coronary sinus, superior vena cava, or inferior vena cava) so that the outer circumference of the generally circular main region 39 is in contact with a circumference inside the tubular region. In one embodiment, at least about 50% of the circumference of the generally circular main region is in contact with a circumference inside the tubular region. In another embodiment, at least about 70% of the circumference of the generally circular main region is in contact with a circumference inside the tubular region. In still another embodiment at least about 80% of the circumference of the generally circular main region is in contact with a circumference inside the tubular region.

The circular arrangement of the electrodes 37 on the generally circular mapping assembly permits measurement of the electrical activity at that circumference of the tubular structure so that ectopic beats between the electrodes can be identified. The size of the generally circular main region 39 permits measurement of electrical activity along a diameter of a pulmonary vein or other tubular structure of or near the heart because the circular main region has a diameter generally corresponding to that of a pulmonary vein or the coronary sinus.

Once the generally circular mapping section 17 is placed in the tubular region, a distal force may be exerted on the catheter shaft by pushing the catheter shaft distally. This distal force causes the generally linear mapping section 14 to flop over and contact a linear region L extending from the tubular region T, as shown in FIG. 7. The electrodes 33 mounted on the generally linear mapping section 14 of the mapping assembly 13 can then be used to measure electrical activity along the linear region extending from the tubular region.

If desired, two or more puller wires can be provided to enhance the ability to manipulate the mapping assembly. In such an embodiment, a second puller wire and surrounding second compression coil extend through the catheter body and into the fourth lumen 36 in the generally linear mapping section 14 of the mapping assembly 13. The first puller wire can be anchored proximal to the anchor location of the second puller wire. Suitable designs of catheters having two or more puller wires, including suitable control handles for such embodiments, are described, for example, in U.S. Pat. No. 6,123,699, entitled “Omni-Directional Steerable Catheter,” U.S. Pat. No. 6,171,277, entitled “Bi-Directional Control Handle for Steerable Catheter,” U.S. Pat. No. 6,183,463, entitled “Bi-directional Steerable Catheter with Bi-directional Control Handle,” and U.S. Pat. No. 6,198,974, entitled “Bi-Directional Steerable Catheter,” the entire contents of which are incorporated herein by reference.

The preceding description has been presented with reference to presently preferred embodiments of the invention. Workers skilled in the art and technology to which this invention pertains will appreciate that alterations and changes in the described structure may be practiced without meaningfully departing from the principal, spirit and scope of this invention. Accordingly, the foregoing description should not be read as pertaining only to the precise structures described and illustrated in the accompanying drawings, but rather should be read consistent with and as support to the following claims which are to have their fullest and fair scope.

What is claimed is:

1. A mapping catheter comprising:
a catheter body having proximal and distal ends and at least one lumen extending therethrough; and
a mapping assembly comprising:
a generally linear mapping section comprising a segment of flexible tubing having proximal and distal ends, wherein the proximal end of the tubing is attached to the distal end of the catheter body,
a generally circular mapping section comprising a segment of flexible tubing having a generally straight proximal region attached to the distal end of the generally linear mapping section and a generally circular main region generally transverse and distal to the generally straight proximal region and having an outer circumference, wherein the generally circular mapping section is adapted to sit in an inner circumference of a generally tubular region and the generally linear mapping section is adapted to lie on a generally linear region extending from the tubular region.

2. The mapping catheter according to claim 1, further comprising at least one electrode mounted on the generally linear mapping section.

3. The mapping catheter according to claim 2, wherein the at least one electrode comprises a plurality of electrodes, the number of electrodes ranging from six to twenty.

4. The mapping catheter according to claim 2, wherein the at least one electrode comprises a plurality of electrodes, the number of electrodes ranging from eight to twelve.

5. The mapping catheter according to claim 2, wherein the at least one electrode comprises ten electrodes.

6. The mapping catheter according to claim 2, further comprising at least one electrode mounted on the generally circular mapping section.

7. The mapping catheter according to claim 1, wherein the generally linear mapping section comprises a material having a durometer ranging from about 40 to about 65D.

8. The mapping catheter according to claim 1, wherein the generally linear mapping section comprises a compound mixture of 55D polyurethane, 65D polyurethane and 80A polyurethane.

9. The mapping catheter according to claim 1, further comprising an electromagnetic sensor mounted in the generally linear mapping section of the mapping assembly.

10. The mapping catheter according to claim 1, further comprising a control handle mounted at the proximal end of the catheter body and means for deflecting the generally linear mapping section by manipulation of the control handle.

11. The mapping catheter according to claim 10, wherein the means for deflecting comprises a puller wire having proximal and distal ends, the puller wire extending from the control handle, through the catheter body and into a lumen in the generally linear mapping section of the mapping assembly, wherein the distal end of the puller wire is fixedly secured within the generally linear mapping section and the proximal end of the puller wire is fixedly secured within the control handle, whereby manipulation of the control handle moves the puller wire relative to the catheter body, resulting in deflection of the generally linear mapping section.

12. A mapping catheter comprising:
a catheter body having proximal and distal ends and at least one lumen extending therethrough; and
a mapping assembly comprising:
a generally linear mapping section comprising a segment of flexible tubing having proximal and distal ends, wherein the proximal end of the tubing is attached to the distal end of the catheter body;
a generally circular mapping section comprising a segment of flexible tubing having a generally straight proximal region attached to the distal end of the generally linear mapping section and a generally circular main region generally transverse and distal to the generally straight proximal region and having an outer circumference, wherein the generally linear mapping section is more flexible than both the catheter body and generally circular mapping section;
at least one electrode mounted on the generally linear mapping section; and
at least one electrode mounted on the generally circular mapping section.

13. The mapping catheter according to claim 12, wherein the at least one electrode mounted on the generally linear mapping section comprises a plurality of electrodes, the number of electrodes ranging from six to twenty.

14. The mapping catheter according to claim 12, wherein the at least one electrode mounted on the generally linear mapping section comprises a plurality of electrodes, the number of electrodes ranging from eight to twelve.

15. The mapping catheter according to claim 12, wherein the at least one electrode mounted on the generally linear mapping section comprises ten electrodes.

16. The mapping catheter according to claim 12, wherein the generally linear mapping section comprises a material having a durometer ranging from about 40 to about 65D.

17. The mapping catheter according to claim 12, wherein the generally linear mapping section comprises a compound mixture of 55D polyurethane, 65D polyurethane and 80A polyurethane.

18. A method for mapping electrical activity within a tubular region of or near the heart, the method comprising:
inserting into the heart the distal end of the catheter according to claim 1;
contacting the outer circumference of the generally circular main region of the generally circular mapping region of the mapping assembly with an inner circumference of the tubular region;
exerting distal force on the catheter body to thereby cause the generally linear mapping section to flop over and contact a linear region of the heart extending from the tubular region;
mapping the electrical activity within the tubular region with the at least one electrode along the generally circular main region of the generally circular mapping section of the mapping assembly; and
mapping the electrical activity of the linear region with the at least one electrode along the generally linear mapping section of the mapping assembly.

19. The method according to claim 18, wherein the at least one electrode mounted on the generally linear mapping section comprises a plurality of electrodes, the number of electrodes ranging from six to twenty.

20. The method according to claim 18, wherein the at least one electrode mounted on the generally linear mapping section comprises a plurality of electrodes, the number of electrodes ranging from eight to twelve.

21. The method according to claim 18, wherein the at least one electrode mounted on the generally linear mapping section comprises ten electrodes.

22. The method according to claim 18, wherein the generally linear mapping section comprises a material having a durometer ranging from about 40 to about 65D.

23. The method according to claim 18, wherein the generally linear mapping section comprises a compound mixture of 55D polyurethane, 65D polyurethane and 80A polyurethane.
